(19) United States
(12) Patent Application Publication
(10) Pub. No.: US 2008/0275369 A1
(43) Pub. Date: Nov. 6, 2008

(54) Arrangement and Method for Determining Muscular Contractions in an Anatomical Organ

(76) Inventor: Lars Fandriks, Askim (SE)

Correspondence Address: Schwegman, Lundberg, Woessner & Kluth, P.A., PO Box 2938, Minneapolis, MN, 55402, US

(21) Appl. No.: 11/547,434

(22) PCT Filed: Mar. 24, 2005
(86) PCT No.: PCT/SE2005/000434

§ 371 Date: Sep. 29, 2006

(30) Foreign Application Priority Data
Mar. 30, 2004 (SE) 0400817-3

(51) Int. Cl. A61B 5/103

(52) U.S. Cl. 600/593

(57) ABSTRACT

The invention relates to an arrangement for determining the degree of muscular contraction of a generally tubular anatomical organ (4), said arrangement comprising a longitudinally extending catheter (9) being adapted for introducing into said organ (4), and a measuring unit (11; 12) for cooperating with said catheter (9) and being adapted for assessing said degree of contraction. According to the intention, said catheter (9) is provided with a sensor arrangement (10; 22; 25) for indicating the geometrical configuration of said catheter (9), and said measuring unit (11; 12) is provided with means for determining the degree of longitudinal muscular contraction of said organ (4) based on values indicating said geometrical configuration. The invention also relates to a catheter for of generally longitudinal extension and being adapted for introducing into a generally tubular anatomical organ. The invention also relates to a method for assessing the degree of said muscular contraction.

### TECHNICAL FIELD

The present invention relates to an arrangement for determining the degree of muscular contraction of a generally tubular anatomical organ, said arrangement comprising a longitudinally extending catheter being adapted for introducing into said organ, and a measuring unit for cooperating with said catheter and being adapted for assessing said degree of contraction.

The invention also relates to a catheter of generally longitudinal extension and being adapted for introducing into a generally tubular anatomical organ.

The invention also relates to a method for determining the degree of muscular contraction of a generally tubular anatomical organ, said method comprising introducing a longitudinally extending catheter into said organ, and assessing said degree of contraction by means of a measuring unit cooperating with said catheter.

### STATE OF THE ART

The main functions of the gastrointestinal system are to digest ingested food and to absorb needed nutrients. The gastrointestinal tract is principally a two-layer muscular tube consisting of one inner layer with the muscular fibres oriented in the circumferential direction and one outer layer with muscular fibres oriented in the axial direction. The inside of the tube is covered by a mucosa that contributes with digestive factors and executes the absorptive function.

The mechanical functions due to activity in these muscular layers differ somewhat along the gut; the first part; i.e. the oesophagus, being primarily transporting; the stomach acts as a reservoir and has a grinding function as well as transporting properties. The latter function is subjected to precise regulation in relation to the digestive capacity in order to deliver matched portions of chyme from the stomach and into the small intestine.

Small intestinal muscular activity is primarily regulated to obtain an optimal exposure of the luminal contents to the absorbing mucosal surface together with a gradual linear transport. In continuation of the small intestine is the large bowel that, in addition to transport, has a prominent reservoir function coupled to a regulated evacuation behaviour of luminal contents that has not been absorbed. The mechanical activity of the gut tube is extremely well-coordinated by use of both enteric nerves and blood-borne signalling compounds.

It is well known that contractions of the circular muscular layer (i.e. consisting of the above-mentioned muscular fibres oriented in the circumferential direction) of the generally tubular organs in the gastrointestinal system create pressure gradients that mix and propel the luminal contents. Looked upon from a functional point of view, the circular muscular layer acts as a peristaltic pump. The function of the longitudinal muscular layer (i.e. consisting of the above-mentioned muscular fibres oriented in the axial direction) of the gastrointestinal system, on the other hand, is less obvious and has because of lack of appropriate methodology not been as extensively studied as the circumferential activity.

An axial shortening of the gut may be considered to contribute to propulsion by creating components of a piston pump where the gut wall acts as a moving cylinder and the luminal contents acts as a passive piston. Integrated with circular peristaltic activity, such mechanical events may create complex and effective forces for propulsion and mixing. However, these conditions have not been possible to study in detail in vivo in man. As a consequence, there is a need for arrangements and methods by means of which the movements of the gut wall can be more accurately measured and determined.

Symptoms of disturbed gut functions are very common. However, only a minority of the individuals that consult medical services are diagnosed as having an organic disease. It follows that most cases with gastrointestinal complaints will be categorized into what is called “functional disorders” of the gut. This means that the employed diagnostic procedures have failed to point out a structural pathological focus. Although the pathophysiology behind functional disorders of the gut is obscure, a common view is that a dysfunctional gastrointestinal motor function causes the distressing symptoms.

It is previously known how to assess gastrointestinal muscular activity in-vivo by several principally different methods; transit time for luminal transport utilizes markers that are ingested and followed; intraluminal hydrostatic pressure gradients can be measured by intraluminal multiple manometry and volume-pressure relationships can be plotted by use of balloons located in various positions within the gut. However, generally all previously known methods for the assessment of gastrointestinal motility, or muscular activity, has a bad association to functional symptoms.

For example, functional dyspepsia, which is defined as unpleasant sensations supposed to originate from the upper gut, has a relation to altered gastric emptying rate but only in less than 50% cases. Furthermore, although other means of investigating local motility patterns, by e.g. barium contrasted x-ray or ultrasound examinations, in some cases appear to reveal a casual relationship between motor actions and symptoms, such methods are not to be regarded as routine diagnostics because they are impractical and often not well tolerated by the patient. In addition, such methods give an on-the-spot reflection that can miss an aberration that occurs over time. Therefore, real time measurement of intra-luminal hydrostatic pressure along the gastrointestinal axis has become a more commonly used technique. A flexible tube is then introduced into the gastrointestinal lumen. Manometric sensors are located at fixed positions along the tube and the pressures are recorded and displayed over time.

The technical principles for intra-intestinal manometry are based on the fact that pressure can be sensed directly by e.g. strain gauges or optical fibres included in the intraluminal tube and connected to amplifiers on the outside of the body. Alternatively (and commonest) the tube is supplied with multiple channels, each ending in a sidehole on a fixed position. Water is flowing at a constant and slow rate through the side hole into the intestinal lumen. Hydrostatic pressure in the flow line is measured on the outside of the body. Pressure alterations in this low-compliance flow system are thus dependent on changes in flow resistance which in turn will depend on the hydrostatic pressure that is present in the gastrointestinal lumen.

It should be noted that oscillations of intraluminal pressure in the gut occur as a function of circumferential contractions. Fixed-position manometry therefore reflects activity in the circular muscular layer. However, as mentioned above, there is a need for arrangements and methods by means of which the gastrointestinal motility can be assessed in a more accurate manner. This is a problem, in particular since the previously known methods for measuring gastrointestinal motility are either too impractical, too inaccurate or too costly.

### SUMMARY OF THE INVENTION

An object of the present invention is to provide an arrangement and a method for assessing muscular contractions in generally tubular anatomical organs, in particular for measuring the gastrointestinal motility, and by means of which the above-mentioned problem can be solved.

This object is accomplished by means of an arrangement of the type as mentioned initially, which is further characterized in that said catheter is provided with a sensor arrangement for indicating the geometrical configuration of said catheter, and that said measuring unit is provided with means for determining the degree of longitudinal muscular contraction of said organ based on values indicating said geometrical configuration.

Said object is also accomplished by means of a catheter as mentioned initially, which is provided with a sensor arrangement for indicating the geometrical configuration of said catheter.

Said object is also accomplished by means of a method of the type as mentioned initially, said method further comprising: detecting values related to the geometrical configuration of said catheter by means of a sensor arrangement provided on said catheter, feeding said values to said measuring unit, and determining the degree of longitudinal muscular contraction of said organ based on said values indicating said geometrical configuration.

The present invention relates to an arrangement and a method for assessing axial movements in certain parts of the gut, for example the duodenum or the colon. Preferably, information related to said axial movement can be combined with other (e.g. manometric) recordings for assessing circumferential contractions of such parts of the gut.

Previously known methods for motility measurements do not provide any reliable and useful methodology for simultaneously assessing the circular and longitudinal gut wall contractile activity. In this regard, it is important to note that contractions in the outer longitudinal muscular layer do not create pressure alterations and can thus not be assessed as such by conventional manometry.

Consequently, an important advantage of the invention is that it allows a combined assessment and display of both circumferential and longitudinal motor actions. For this reason, such a combined display will exhibit novel insights into the complex basis for symptom generation following gut motor disorders.

A further advantage of the present invention is that it gives a possibility to develop medical drugs which focus on the longitudinal muscular layer. Today, such drugs cannot be developed due to the fact that methods and devices for measuring the effect of such drugs on the longitudinal muscular layers and their motility do not exist.

### DESCRIPTION OF THE DRAWINGS

The invention will now be described in greater detail with reference to certain embodiments and the appended drawings, in which:

FIG. 1a discloses a part of the human gastro-intestinal system in a first, relaxed, condition;

FIG. 1b discloses a part of the human gastro-intestiotial system in a second, contracted, condition;

FIG. 2 shows a measurement system in accordance with the present invention;

FIG. 3a shows a curvature of a catheter according to the invention in a first condition of the gastrointestinal system; and

FIG. 3b shows a curvature of a catheter according to the invention in a second condition of the gastrointestinal system,

FIG. 4 shows a measurement system in accordance with a second embodiment of the present invention,

FIG. 5 shows a measurement system in accordance with a third embodiment of the present invention; and

FIG. 6 shows a measurement system in accordance with a fourth embodiment of the present invention

### PREFERRED EMBODIMENTS

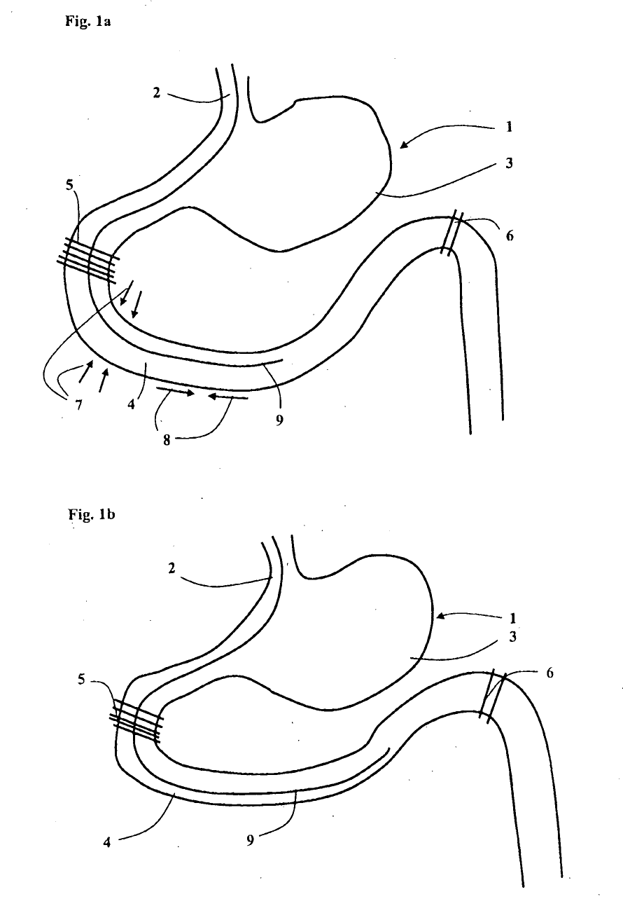

FIGS. 1a and 1b are schematical and simplified drawings of a part of the gastrointestinal system 1 of a human, in two different conditions. According to a preferred embodiment, the present invention relates to an arrangement and a method for assessing longitudinal contractive movements in the form of muscular contractions in anatomical organs of generally tubular shape. In particular, the invention is intended to be used for assessing muscular contraction in the organs of the gastrointestinal system of humans or animals.

According to a first embodiment which will be described below, the invention can be used for assessing the longitudinal contractive movements in the deodenum of a human. However, the invention is not limited to the human gastrointestinal system, but can also be applied in similar measurements in certain types of animals (for example pigs, dogs, cats etc.) having a anatomical structure of the gastrointestinal system which generally corresponds to the description below.

FIG. 1a shows a part of the upper gastrointestinal system of a human and in a relaxed condition, whereas FIG. 1b shows the same part of the upper gastrointestinal system in a contracted condition. As indicated in FIG. 1a, the gastrointestinal system 1 generally comprises the oesophagus 2, which ends in the stomach 3. The stomach 3 is followed by the duodenum 4 which extends in a curve-like manner from the lower section of the stomach 3.

As indicated schematically in FIG. 1a, the duodenum 4 can be said to be fixed in one point, or rather one area The fixing area 5 for the duodenum 4 is defined due to the fact that a descending part of the duodenum 4 (being formed as a continuation of the stomach 3) is fixed retroperitoneally, thereby forming a first anatomically fixed area indicated by means of reference numeral 5 in FIGS. 1a and 1b. Furthermore, a second anatomically fixed area 6 is defined due to the fact that the duodenum 4 is suspended by means of the so-called Treitz ligament. This area is indicated by means of reference numeral 6 in FIGS. 1a and 1b.

As indicated in FIG. 1a by means of a first set of arrows 7, and as explained initially, the duodenum 4 functions so as to contract in a circumferential direction. For this reason, the first set of arrows 7 are arranged in a direction which is generally transversal to the extension of the duodenum 4. Such circular contractions result in pressure gradients in the duodenum 4 that mix and propel the luminal contents. Furthermore, as indicated in FIG. 1a by means of a second set of arrows 8, and as explained initially, the duodenum 4 also functions so as to contract in a generally longitudinal direction along the duodenum 4. This axial shortening of the duodenum, which is due to activity in longitudinally extending muscular fibres, can be assumed to contribute to the propulsion of the luminal contents.

FIG. 1a indicates the duodenum 4 in a first condition which more precisely is a relaxed condition, i.e. a relaxed condition of the longitudinally extending muscular layers in the duodenum 4. In contrast to this first condition, FIG. 1b indicates in a schematical manner the duodenum 4 in a second condition, more precisely a contracted condition. The contracted condition occurs as a result of an axial shortening of the duodenum 4 due to contraction of said longitudinally oriented muscular layer.

As already mentioned, there is a demand for methods and devices for measuring and evaluating the degree of such axial, or longitudinal, contractions of the gastrointestinal system, for example in the duodenum 4 as shown in FIG. 1b. Such measurements would then be expected to provide valuable information regarding research on gastrointestinal motor disorders and would also be used during treatment of related gastrointestinal diseases. To this end, the present invention is based on the principle that an intraluminal catheter 9 can be used in order to estimate gut wall longitudinal movements.

With reference to FIGS. 1a and 1b, the intraluminal catheter 9 is shown in a simplified manner in which it extends through the oesophagus 2, the stomach 3 and duodenum 4. This is the actual positioning of the catheter 9 during measurements by means of the present invention. As can be noted by comparing FIGS. 1a and 1b, the catheter 9 will assume a different configuration during the relaxed state (FIG. 1a) as compared with its configuration during the contracted state (FIG. 1b). The present invention is based on the principle that the physical shape, i.e. the geometrical configuration of the catheter 9, will be determined during the relaxed and contracted state, respectively. Information related to the configuration of the catheter 9 will then be used in assessing the degree of contraction of the duodenum 4. This principle will now be described in greater detail.

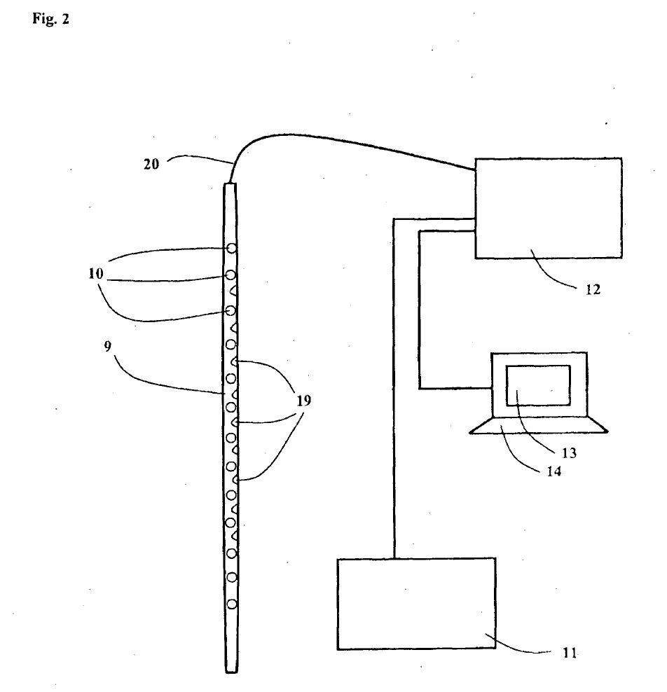

With reference to FIG. 2, there is shown a measuring system comprising the intraluminal catheter 9 as shown in FIGS. 1a and 1b. In FIG. 2, the catheter 9 is shown in a more detailed manner than the more schematical drawings in FIGS. 1a and 1b. In particular, the catheter 9 is constituted by a flexible, generally tubular component having dimensions which are chosen so as allow the catheter 9 to be introduced into the gastrointestinal lumen of a human or an animal. More precisely, and with reference to FIGS. 1a and 1b, the catheter is arranged so as to be introduced into the duodenum 4 via the oesophagus 2 and the stomach 3. Furthermore, the catheter 9 has a length which is sufficient for it to extend along a substantial portion of the duodenum 4, between the two anatomically fixed points, or areas, 5, 6.

As regards suitable materials, the intraluminal catheter 9 is manufactured from a material having a low rigidity and which is capable of being introduced into the gastrointestinal system. Preferably, a plastic or rubber material is used.

In FIG. 2, the catheter 9 is shown in a condition in which it is not introduced into a gastrointestinal system. In accordance with the invention, the catheter 9 is of longitudinal extension and comprises a sensor arrangement adapted for capturing the geometrical configuration of the catheter 9 inside the duodenum 4. According to a first embodiment of the invention, which is shown in FIG. 2, said sensor arrangement is constituted by a plurality of radiologically opaque markers 10 which are positioned along the catheter 9. These markers 10 are adapted so as to be observable, i.e. visualized, by means of fluoroscopy, which is a technique for obtaining x-ray images of a human or an animal. According to this embodiment, the catether 9 with its markers 10 are used in connection with a fluoroscopy scanning device 11 which is adapted for detecting the position of each of said markers 10. The flouroscopy scanning device 11 forms part of a measuring unit which also comprises a central control unit 12, which preferably is computer-based but which can also be implemented in other ways.

During use of the catheter 9, it will be introduced in the gastrointestinal system as exemplified by the duodenum 4 as shown in FIGS. 1a and 1b. This means that the markers 10 will be detectable by means of the x-ray device 11. In particular, the position of each of the markers 10, for example with reference to an orthogonal coordinate system, will be determined. Data related to the positions of each of the markers 10 will be transferred to the control unit 12 for further evaluation. Based on information from the x-ray device 11, the control unit 12 is adapted for determining the degree of longitudinal contraction of the duodenum 4 based on values indicating the geometrical configuration as provided by the markers 10. In other words, the (control unit 12 is adapted for assessing the geometrical changes in the configuration of the catheter 9 when the gastrointestinal system goes from a relaxed to a contracted condition.

Furthermore, information regarding the configuration of the catheter 9 can be displayed on a display 13 forming part of a computer 14. This means that the control unit 12 and computer 14 with its display 14 and associated software together form part of a measuring unit which can be used for analysis of altered catheter-curvatures including estimation of changes in the axial direction of the gut wall.

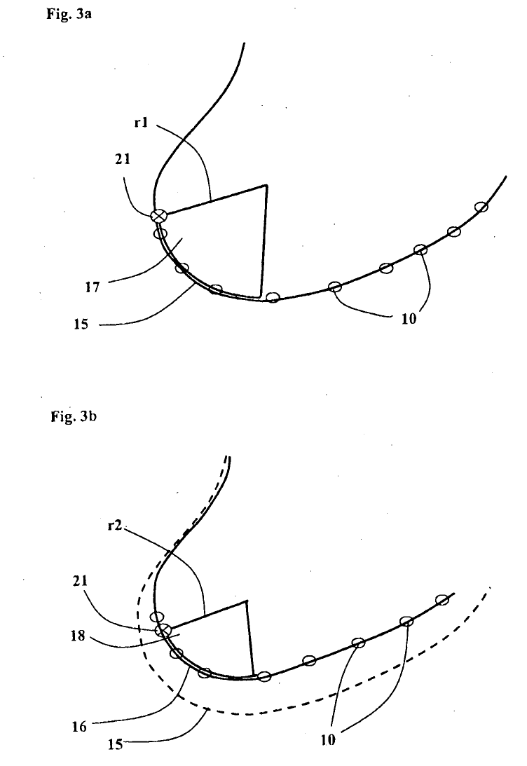

The principles behind the invention will now be described in further detail with reference to FIGS. 3a and 3b. FIG. 3a shows in a simplified and schematical form a first curvature 15 of the catheter 9, as represented by the plurality of markers 10 disposed along the length of the catheter 9. Furthermore, the curvature shown in FIG. 3a corresponds to the relaxed state of the duodenum (cf. FIG. 1a).

On the other hand, FIG. 3b shows a second curvature 16 of the catheter 9 which corresponds to a contracted state of the duodenum (cf. FIG. 1b). For clarity, the above-mentioned first curvature 15 is also shown in FIG. 3b, as a reference. In the contracted state of the duodenum, the curvature of the catheter 9 will be more sharp. By means of the invention, a measure of the degree of contraction is obtained by choosing suitable geometrical parameters which define the amount of contraction of the catheter 9 and consequently also of the duodenum.

The basis for the invention is that during longitudinal relaxation a mobile segment of the duodenum 4 tends to form a curve starting at the anatomically fixed area 5. When an axial shortening occurs due to contraction of the longitudinally oriented muscular layer, this curvature will become more pronounced, as shown in FIG. 3b. It follows that the shape of the intraluminally situated flexible catheter 9 will attain and follow changes in the luminal extension.

Recording of the shape of the catheter 9 makes it possible to calculate the radius of a hypothetical circle which fit to the curvature under study. The proportionality of the radius to the circumference is useful as an absolute measure of longitudinal movements in relation to the fixed part of duodenum 4.

As indicated in FIG. 3a, a circular segment 17 representing the curvature of the catheter 9 is fitted into the curvature 15 representing the relaxed condition of the duodenum 9. In a similar manner, as indicated in FIG. 3b, a further circular segment 18 representing the curvature of the catheter 9 in the contracted condition is fitted into the curvature 15. Furthermore, in FIGS. 3a and 3b, the circular segments 17, 18 extend from a fixed reference point 21 which is the same in both the relaxed and the contracted state of the duodenum, and which generally correspond to the above-mentioned first fixing area 5.

Preferably, the invention is arranged so that: the circular segments 17, 18 are used so as to calculate an imaginary circumference for both the relaxed and the contracted condition of the duodenum. Due to the fact that the radius r1 of the relaxed state (FIG. 3a) and the radius r2 of the contracted state (FIG. 3b) are indicated, values of the corresponding circumferences can be calculated using the well-known formula Z*π*r. The calculated values of the two circumferences are then used to assess the degree of longitudinal contraction of the duodenum.

Alternatively, more than one curvatures can be defined along the extension 15, 16 of the catheter 9. The principles of this embodiment are not shown in the drawings. For example, a first curvature can be defined before the first fixing area 5, a second curvature can be defined immediately after the first fixing area 5 and a third, more flat curvature closer to the second fixing area 6. This will further increase the possibility for even more accurate assessment of the longitudinal contractions of the duodenum over time.

The invention is not limited to the above-mentioned method of determining circular segments as indications of the geometrical configuration. Generally, any method for determining the position, inclination, curvature and shape of the catheter 9 along its extension can also be used in order to determine the degree of longitudinal contraction of the duodenum.

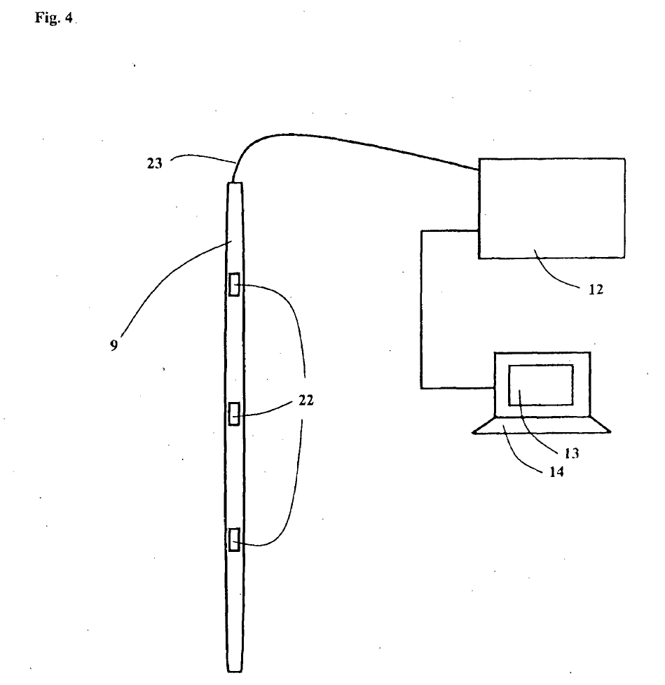

According to a further embodiment of the invention, which is shown in FIG. 4, the catheter 9 can be provided with a sensor arrangement in the form of strain gauges 22 which are sensor devices which are previously known per se. Such sensors can be used in the context of the present invention for providing measurements of the bending of the catheter 9, i.e. measurements of the geometrical configuration of the catheter 9.

Strain gauges can be configured in a Wheatstone bridge, which is an electrical network having four resistive elements. One or several of these elements can be constituted by strain gauges. If bending strain acts upon any one of these strain gauges, the resistance of this gauge will change. By means of the Wheatstone bridge configuration, the variations in resistance generated by the strain gauges can be measured.

In accordance with the principles of the present invention, a number of such strain gauges can be arranged along a catheter. With reference to FIG. 4, three such strain gauges 22 can be positioned along the length of the catheter 9. Each of the gauges 22 is electrically connected as a part of a Wheatstone bridge (not shown), for providing measurements reflecting the bending strain acting upon the catheter during use, and consequently also reflecting the geometry of the catheter 9. Such Wheatstone bridges can be integrated into a control unit 12, which means that the strain gauges 22 are connected to said control unit 12 via an electrical connection 23.

In a manner which is analog to that of FIG. 2, the embodiment according to FIG. 4 can be adapted so that a computer 14 is used for determining the degree of longitudinal muscular contraction of the duodenum based on values indicating the geometrical configuration as provided by the strain gauges 22. Additionally, the geometrical configuration of the catheter 9 can be displayed on a display 13 cooperating with the computer 14.

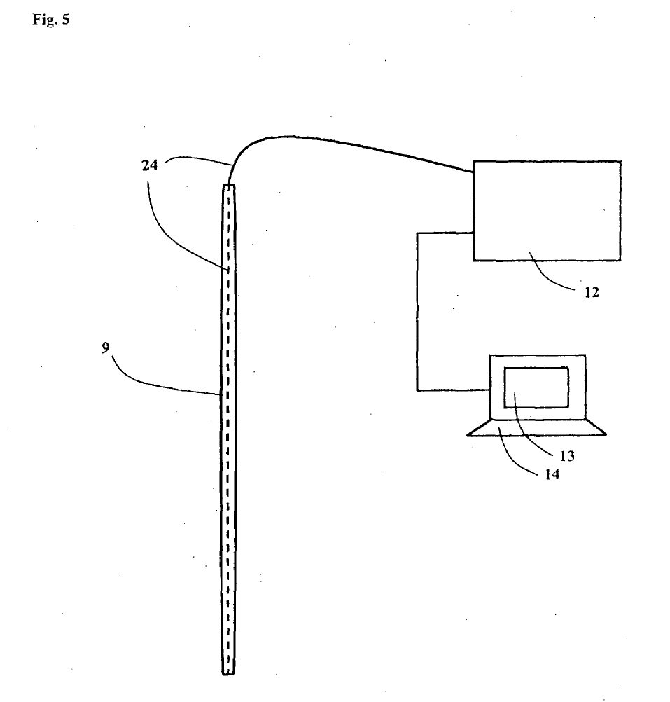

According to a further embodiment, which is shown in FIG. 5, the catheter 9 can be provided with a sensor arrangement in the form of one or more optical fibers. Said fiber or fibers are schematically indicated as one single fiber having reference numeral 24 in FIG. 5. Preferably, the fiber 24 extends along the inside of the catheter 9 so as to follow the curvature of the catheter 9. Furthermore, it is previously known to provide an optical fiber bend sensor for measuring the degree and orientation of bending of said fiber. The degree of bending can for example be assessed by detecting an interference pattern resulting from light propagating through such a fiber or fibers and being modulated as a result of bending of the fiber. To this end, the fiber 24 can for example comprise bending-sensitive elements (not shown) which can be positioned in the catheter 9 so as to reflect any bending thereof. In such a manner, the degree and orientation of bending which is present in the fiber 24 (and consequently also in the catheter 9) can be assessed. More precisely, the actual assessment of the signals from the fiber 24 is carried out by means of a central control unit 12, to which the optical fiber 24 is connected, as indicated in FIG. 5. Obviously, the control unit 12 is adapted for converting optical signals suitable for transmitting along the fiber 24 to digital signals suitable for assessment of the actual geometrical configuration of the fiber 24, and vice versa.

Consequently, the fiber 24 according to FIG. 5 is sensitive to external forces, for example caused by the longitudinal muscular contractions as explained above. Such a sensor arrangement can be used in the context of the present invention for detecting the bending of the catheter 9, and consequently for providing measurements of the geometrical configuration of the catheter 9.

Additionally, the geometrical configuration of the catheter 9 can be displayed on a display 13 cooperating with a computer 14 which is connected to the control unit 12.

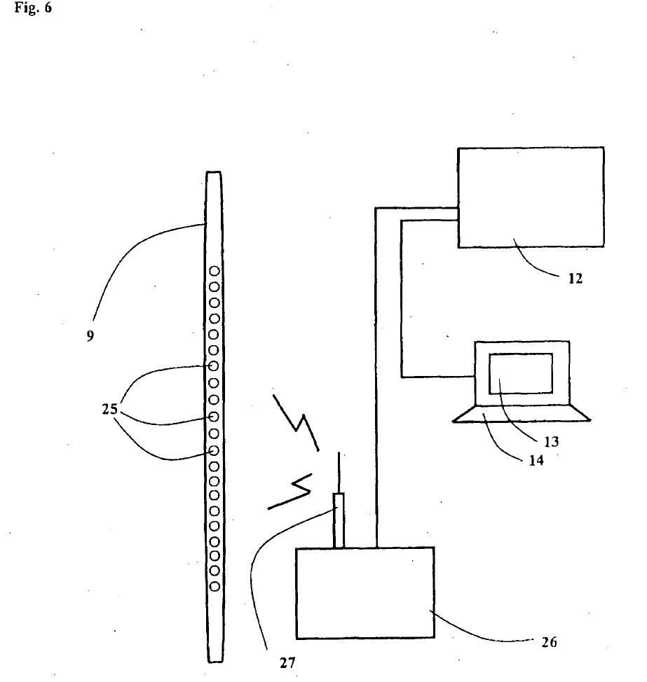

According to a further embodiment, which is shown in FIG. 6, the catheter 9 can be provided with a sensor arrangement in the form of a number of transponders 25 which are adapted for providing information related to their positions and for transmitting said information to an external transmitter/receiver unit 26. To this end, the transmitter/receiver unit 26 is provided with an antenna 27 for communicating with said transponders 26. Also, the transmitter/receiver unit 26 is connected to a control unit 12 which is adapted to assess the actual geometric configuration of the catheter 9 based on information regarding the positions of each of the transponders 24, as captured by the transmitter/receiver unit 26 with its antenna 27.

The transponders 26 can for example be in the form of passive transponder tags which cooperate with the antenna 27 for detecting the position of each such tag. In this manner, such a sensor arrangement can be used in the context of the present invention for providing measurements of the geometrical configuration of the catheter.

Additionally, the geometrical configuration of the catheter 9 can be displayed on a display 13 cooperating with a computer 14, which in turn is connected to the control unit 12.

Preferably, the invention should be combined with other assessments of gut motor activity, e.g. manometry or recordings of flow of luminal contents (e.g. by impedance measurements) or other functional assessments (e.g. recording of transmucosal potential difference).

The possibility of combining the inventive assessment of longitudinal muscular activity with manometry measurements is indicated schematically in FIG. 2, which shows that the catheter 9 is provided with means for manometry measurements by means of a number of side-holes 19. In this manner, manometric sensors are located at fixed positions along the tube and the pressures are recorded and displayed over time. To this end, the manometric sensors are connected to the control unit 12 by means of a connection 20. Such a multilumen manometric catheter is used to record changes in intraluminal pressure due to circumferential contraction caused by activity in the gastrointestinal circular muscular layer. The principles for such manometric measurements are previously known per se. For this reason, they are not described in any detail here. However, it can be noted that a combined assessment of both circumferential and longitudinal contractions of the duodenum can be expected to provide new insights into gastrointestinal disorders.

For reasons of simplicity, the catheter according to the embodiments shown in FIGS. 4, 5 and 6 is shown without any sideholes of the kind as shown in FIG. 2, which are used for manometry measurements. However, it should be noted that the embodiments according to FIGS. 4-6 can also be provided with such means for manometry. In this manner, the longitudinal and circumferential contractions of the duodenum can be measured simultaneously in all the embodiments of the invention.

According to its basic principle, the invention describes a device that estimates gut wall longitudinal movements by assessing and displaying the three dimensional shape of an intraluminal flexible catheter positioned at certain locations along the intestinal extension.

The invention can be used for assessing muscular contractions in various parts of the gastrointestinal system, for example the duodenum, but for example also the colon. The invention is applicable at certain regions of the gut that can be reached by an intubation procedure and where there exist mobile parts of the gut as well as a part fixed to known anatomical positions. An example of such anatomical fixation points of the gut are the is descending part of the duodenum with both the proximal and distal part as mobile parts, as shown in FIGS. 1a and 1b. Another example is the ascending and descending large bowel with the transverse colon being mobile (not shown). A third example is the anorectal association to the pelvic floor with sigmoid colon as mobile part.

The invention is not limited to the above-mentioned embodiment but can be varied within the scope of the appended claims. For example, the measurements of the longitudinal contractions can be carried out independently of the measurements of the circumferential contractions. However, it is advantageous to combine these two measurements, in particular by providing the catheter with sensors for carrying out both these types of measurements.

The arrangement, method and catheter according to the invention can be used for measurements related to both humans and animals.

Finally, the invention can be used in connection with other generally tubular anatomical organs than those of the gastrointestinal system. For example, the invention can be used for measurements in the ducts of the biliary or urogenital tracts.

1. Arrangement for determining the degree of muscular contraction of a generally tubular anatomical organ (4), said arrangement comprising:
a longitudinally extending catheter (9) being adapted for introducing into said organ (4), and a measuring unit (11; 12) for cooperating with said catheter (9) and being adapted for assessing said degree of contraction, characterized in that said catheter (9) is provided with a sensor arrangement (10; 22; 25) for indicating the geometrical configuration of said catheter (9), and that said measuring unit (11; 12) is provided with means for determining the degree of longitudinal muscular contraction of said organ (4) based on values indicating said geometrical configuration.

2. Arrangement according to claim 1,
characterized in that said sensor arrangement (10) comprises a plurality of radiologically opaque markers (10) arranged on said catheter (9), and in that said measuring unit (11; 12) comprises a fluoroscopy scanning device (11) adapted for detecting the positions of said markers (10).

3. Arrangement according to claim 1,
characterized in that said sensor arrangement (10) comprises a plurality of strain gauges (22) arranged in the catheter (9) for detecting bending strain acting upon said catheter (9).

4. Arrangement according to claim 1,
characterized in that said sensor arrangement (10) comprises at least one optical fiber (24) being adapted for detecting bending thereof.

5. Arrangement according to claim 1,
characterized in that said sensor arrangement (10) comprises a plurality of transponders (25) which cooperate with a sender/receiver arrangement (26, 27) for providing information regarding the position of each transponder.

6. Arrangement according to claim 1, characterized in that said catheter (9) also comprises means (19) for side-hole manometry in said organ (4), for assessing circumferential muscular contractions of said organ (4).

7. Arrangement according to claim 1, characterized in that it is adapted for determining the degree of contraction of muscular contraction of a generally tubular organ (4) in a gastrointestinal system of a human or animal.

8. Catheter (9) of generally longitudinal extension and being adapted for introducing into a generally tubular anatomical organ (4), characterized in that said catheter (9) is provided with a sensor arrangement (10; 22; 25) for indicating the geometrical configuration of said catheter (9).

9. Catheter (9) according to claim 8,
characterized in that said sensor arrangement comprises a plurality of radiologically opaque markers (10).

10. Catheter (9) according to claim 8,
characterized in that said sensor arrangement comprises a plurality of strain gauges (22) for detecting bending strain acting upon said catheter (9).

11. Catheter (9) according to claim 8,
characterized in that said sensor arrangement comprises at least one optical fiber (24) being adapted for detecting bending thereof.

12. Catheter (9) according to claim 8,
characterized in that said sensor arrangement comprises a plurality of transponders (25) adapted to cooperate with a sender/receiver arrangement (26, 27) for providing information regarding the position of each transponder.

13. Catheter (9) according to claim 8,
characterized in that said catheter (9) also comprises means (19) for side-hole manometry in said organ (4).

14. Method for determining the degree of muscular contraction of a generally tubular anatomical organ (4), said method comprising:
introducing a longitudinally extending catheter (9) into said organ (4), and
assessing said degree of contraction by means of a measuring unit (11; 12) cooperating with said catheter (9), characterized in that said method further comprises:
detecting values related to the geometrical configuration of said catheter (9) by means of a sensor arrangement provided on said catheter (9),
feeding said values to said measuring unit (11; 12), and
determining the degree of longitudinal muscular contraction of said organ (4) based on said values indicating said geometrical configuration.

15. Method according to claim 14,
characterized in that the step of detecting values related to the geometrical configuration of said catheter (9) is carried out by means of a plurality of radiologically opaque markers (10) arranged on said catheter (9), and by detecting the positions of said markers (10) by means of a fluoroscopy scanning device (11).

16. Method according to claim 14,
characterized in that the step of detecting values related to the geometrical configuration of said catheter (9) is carried out by detecting values representing bending strain acting upon said catheter (9) by means of a plurality of strain gauges arranged in the catheter (9).

17. Method according to claim 14,
characterized in that the step of detecting values related to the geometrical configuration of said catheter (9) is carried out by detecting bending of at least one optical fiber (24) being arranged in said catheter (9).

18. Method according to claim 14,
characterized in that the step of detecting values related to the geometrical configuration of said catheter (9) is carried out by providing information regarding the position of a plurality of transponders (25) arranged on said catheter (9) and cooperating with a sender/receiver arrangement (26, 27).

19. Method according to claim 14,
characterized in that it furthermore comprises detecting circumferential muscular contractions in said organ (4) by means of side-hole manometry means (19).

20. Method according to claim 14,
characterized in that it comprises determining the degree of muscular contraction of a generally tubular organ (4) in a gastrointestinal system of a human or animal.

21. Method according to claim 14,
characterized in that it comprises determining said geometrical configuration with reference to at least one anatomically fixed reference points (5, 6).
